(19) United States
(12) Patent Application Publication
(10) Pub. No.: US 2022/0346816 A1
(43) Pub. Date: Nov. 3, 2022

(54) FORCE LIMITING MECHANISM FOR SURGICAL INSTRUMENTS

(71) Applicant: Conmed Corporation, Largo, FL (US)

(72) Inventor: Aaron Szabo, Swanton, OH (US)

(73) Assignee: Conmed Corporation, Largo, FL (US)

(21) Appl. No.: 17/638,268

(22) PCT Filed: Aug. 25, 2020
(86) PCT No.: PCT/US20/47773

§ 371 Date: Feb. 25, 2022

Related U.S. Application Data
(60) Provisional application No. 62/892,006, filed on Aug. 27, 2019.
(60) Provisional application No. 62/900,104, filed on Sep. 13, 2019.

(51) Int. Cl. A61B 17/29; A61B 90/00

(52) U.S. Cl. CPC A61B 17/2909; A61B 90/03; A61B 2017/292

(57) ABSTRACT

A surgical instrument that limits the amount of force that a user can apply to the jaws of a surgical instrument by decoupling the handle lever from the drive shaft if the handle lever is moved beyond the closed position. A handle lever and a secondary lever are interconnected by a spring that can decouples the handle lever from the secondary lever when too much force is used. The handle lever and the secondary lever may be interconnected by a link pivotally interconnected both the first lever and the second lever, or the handle lever and the secondary lever may be mounted to a common pivot point. In the former, the spring is configured to bias the handle lever and the secondary lever together, and in the latter the spring is configured to bias the handle lever and the secondary lever apart.

### CROSS-REFERENCE TO RELATED APPLICATIONS

The present application claims priority to U.S. Provisional No. 62/892,006, filed on Aug. 27, 2019 and U.S. Provisional No. 62/900,104, filed on Sep. 13, 2019.

### BACKGROUND OF THE INVENTION

### 1. Field of the Invention

The present invention relates to surgical instruments and, more specifically, to a force limiting mechanism for limiting the amount of force applied to the jaws of an electrosurgical instrument.

### 2. Description of the Related Art

Electrosurgical vessel sealers are used for the occlusion of blood vessels and halting of bleeding during surgical procedures. The electrodes of the vessel sealer are carried by a pair of opposing jaws and interconnected to an electrosurgical generator that can selectively supply radiofrequency (RF) energy to the electrodes. A user may close the jaws around a vessel to be sealed by squeezing a lever associated with a handle assembly. The vessel may then be sealed by supplying the RF energy to the clamped vessel. A moveable blade may be additionally incorporated into the jaws for cutting of the sealed blood vessel along an intermediate portion of the seal created by the energized electrodes in response to user activation of a second trigger.

One problem that arises in the use of electrosurgical vessel sealers is the user applying too much force to the jaw closing lever, which can result in breakage of the device. Accordingly, there is a need in the art for an approach that can limit the amount of force that a user can apply to the jaws via the handle lever.

### BRIEF SUMMARY OF THE INVENTION

The present invention limits the amount of force that a user can apply to the jaws of a surgical instrument by decoupling the handle lever from the drive shaft if the handle lever is moved beyond the closed position. More specifically, the present invention is a surgical instrument comprising a body having a drive shaft extending along a longitudinal axis and coupled to a pair of jaws that are moveable between an open and a closed position, a lever bearing secured around the drive shaft for movement therewith and having a stop extending therefrom, a first lever having a first upper end pivotally mounted within the body and a first lower end that extends out of the body, a second lever having a second upper end interconnected to the first upper end to the first lever and extending to a second lower end, wherein the second lever engages the stop of the lever bearing, and a spring extending between an intermediation portion of the first lever and the second lower end of the second lever. The second lever may include a pair of bearing surfaces in contact with the stop of the lever bearing.

In one embodiment, the surgical instrument may further comprise a link pivotally interconnected to the first upper end of the first lever and to the second upper end of the second lever. The second lever may include a pair of bearing surfaces in contact with the stop of the lever bearing. In this embodiment, the spring may be configured to provide a force biasing the first lever and the second lever together. The first lever may be moveable between a first position where the first lever causes the second lever to position that lever bearing so that the drive shaft puts the jaws are in the open position, a second position where first lever causes the second lever to position the lever bearing position so that the drive shaft puts the jaws in the closed position, and a third position where the first lever has separated from the second lever against the bias of the spring so that the second lever and lever bearing do not move the drive shaft and the jaws remain in the closed position. A pair of tabs may extend between a first set of posts positioned on the first lever and a second set of posts positioned in the second lever that set a predetermined minimum distance between the first lever and the second lever.

In another embodiment, the first lever and the second lever may be pivotally mounted within the body about a common pivot point. In this embodiment, the spring is configured to provide a force biasing the first lever and the second lever apart. The first lever is moveable between a first position where the first lever causes the second lever to position that lever bearing so that the drive shaft puts the jaws are in the open position, a second position where first lever causes the second lever to position the lever bearing position so that the drive shaft puts the jaws in the closed position, and a third position where the first lever moved closer to the second lever against the bias of the spring so that the second lever and lever bearing do not move the drive shaft and the jaws remain in the closed position.

In a further embodiment, the present invention may comprise a method of limiting the amount of force applied by a user to the jaws of a surgical instrument. The method includes the steps of providing a first lever extending from a body of the surgical instrument for movement by a user to close the jaws of the surgical instrument, providing a second lever that is responsive to movement of the first lever to translate a lever bearing axially along a longitudinal axis of the body of the surgical instrument, and interconnecting the first lever and the second lever with a spring that allows for movement of the second lever along with the first lever when the first lever is moved to a position that results in closing of the jaws of the surgical instrument but prevents movement of the second lever when the first lever is moved beyond the closing of the jaws the second lever so that movement of the first lever past the position that results in closing of the jaws. The first lever and the second lever may be interconnected a link that is pivotally connected to both the first lever and the second lever, with the spring is configured to bias the first lever and the second lever toward each other. The first lever and the second lever may instead by pivotally mounted in the body to a common pivot point, with the spring configured to bias the first lever and the second lever away from each other.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of an electrosurgical system having a pair of jaws carrying electrodes for electrosurgically treating tissue

FIG. 2 is an isometric view of the components of a handle assembly for an electrosurgical vessel sealer according to the present invention;

FIG. 3 is a front view of the components of a partially disassembled handle assembly for an electrosurgical vessel sealer according to the present invention;

FIG. 4A is a first isometric view of a handle and force limiting mechanism according to the present invention;

FIG. 4B is a second isometric view of a handle and force limiting mechanism according to the present invention;

FIG. 5 is an isometric view of a force limiting mechanism according to the present invention;

FIG. 6 is a front view of the handle assembly when the jaws of the electrosurgical vessel sealer are in the open position;

FIG. 7 is a front view of handle assembly when the jaws of the electrosurgical vessel sealer are in the closed position; and

FIG. 8 is a front view of the handle assembly when the jaws of the electrosurgical vessel sealer are in the closed position and the handle lever has been moved beyond the point necessary to close the laws.

FIG. 9 is an isometric view of the components of a second embodiment of a handle assembly for an electrosurgical vessel sealer according to the present invention;

FIG. 10 is an isometric view of a force limiting mechanism according to a second embodiment of the present invention;

FIG. 11A is a side view of a handle and force limiting mechanism in a first position according to a second embodiment of the present invention;

FIG. 11B is a side view of a handle and force limiting mechanism according to a second embodiment of the present invention;

FIG. 12A is a side view of a handle and force limiting mechanism according to a second embodiment of the present invention;

FIG. 12B is an isometric view of a handle and force limiting mechanism in a second position according to a second embodiment of the present invention;

FIG. 13 is an isometric view of a handle lever and force limiting assembly according to a second embodiment of the present invention;

FIG. 14 is a front view of the handle assembly when the jaws of a second embodiment of the electrosurgical vessel sealer are in the open position;

FIG. 15 is a front view of handle assembly when the jaws of a second embodiment of the electrosurgical vessel sealer are in the closed position; and

FIG. 16 is a front view of the handle assembly when the jaws of a second embodiment of the electrosurgical vessel sealer are in closed position and the handle lever has been moved beyond the point necessary to close the laws.

### DETAILED DESCRIPTION OF THE INVENTION

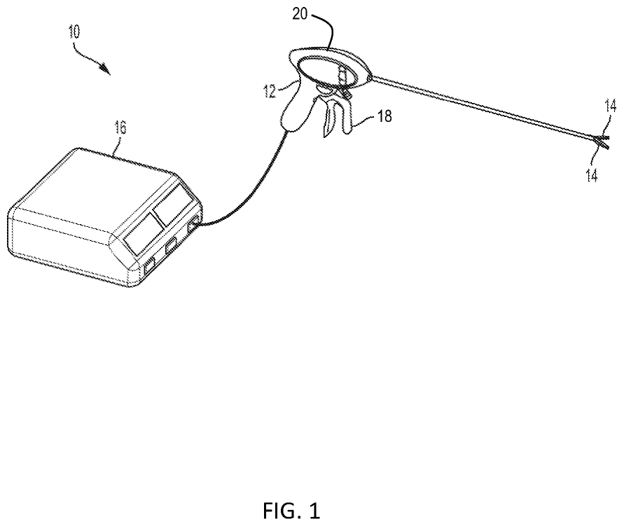

Referring to the figures, wherein like numeral refer to like parts throughout, there is seen in FIG. 1 a vessel sealing system 10 comprising a vessel sealer 12 having a pair of conductive opposing jaws 14 that are interconnected to an electrosurgical generator 16 that can supply RF energy to electrodes of jaws 14 for the desiccation of a blood vessel trapped between jaw 14. The dimensions of jaws 14 and the type of RF energy supplied will produce desiccation of the blood vessel in a region of a particular width as determined by the thermal spread of the energy being supplied to the blood vessel. As is known in the art, jaws 14 are pivotally mounted to vessel sealer 12 for movement between an open position and a closed position in response to a user operating a lever 18 that extends from the main body 22 of sealer 12 and can be gripped by a user.

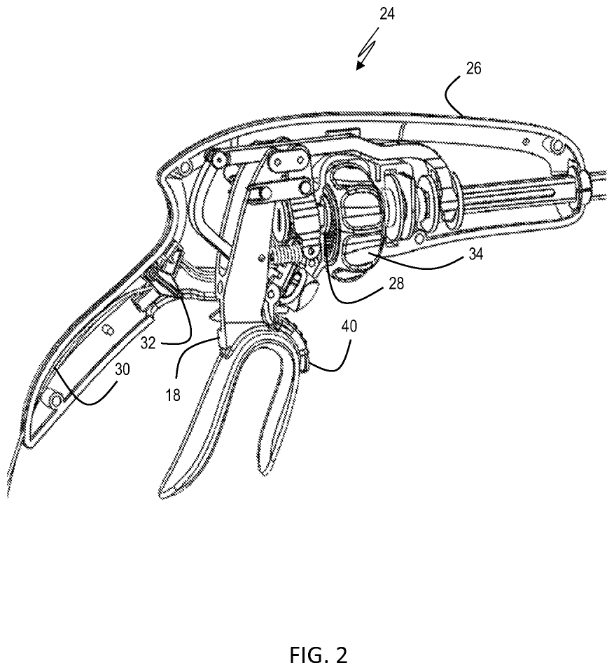

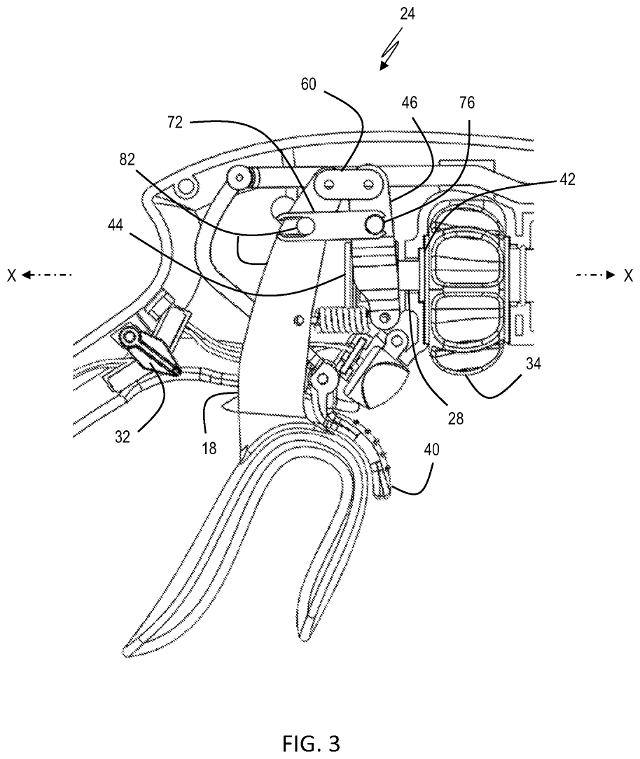

Referring to FIGS. 2 and 3, vessel sealer 12 includes a handle assembly 24 comprised of a housing body 26 that encloses a drive shaft 28 that extends from body 26 and drives a longitudinal axis X-X of vessel sealer 12. Drive shaft 28 is coupled to jaws 14 so that longitudinal movement of drive shaft 28 will mechanically move jaws 14 between the open and closed positioned. Housing body 26 also encloses cabling 30 for delivering energy to jaws 14 as well as a latching mechanism 32 for selectively retaining lever 18 when it is moved from a first position, where jaws 14 are open, to a second position, where jaws 14 are closed. Housing body 26 partially encloses a knob 34 that is coupled to an outer shaft 36 that surrounds drive shaft 28 and supports jaws 14 such that rotation of knob 34 will rotate jaws 14 through 360 degrees. Handle assembly 24 further comprises a knife trigger 40 for extending a blade (not shown) between jaws 14 to sever a treated vessel.

A lever bearing 40 having a distal stop 42 and a proximal stop 44 is secured to drive shaft 28 so that movement of lever bearing 40 causes longitudinal translation of drive shaft 28 and thus opening and closing of jaws 14. Lever bearing 40 is rotatable through 360 degrees along with drive shaft 28 when a user rotates knob 34 so that jaws 14 can be positioned as desired. Lever 18 is interconnected to lever bearing 40 for movement of drive shaft 28 by a secondary lever 46, illustrated as having a H-shaped body with lower legs 48 extending on either side of drive shaft 28 and having proximal bearing surfaces 50 and 52 in engagement with proximal stop 44. Bearing surfaces 50 and 52 will remain in engagement with proximal stop 44 regarding of any rotation of drive shaft 28. As a result, jaws 14 may be closed via operation of lever 18 regardless of any rotation of jaws 14 by knob 34, thereby allowing a user to be able to close jaws 14 in any position.

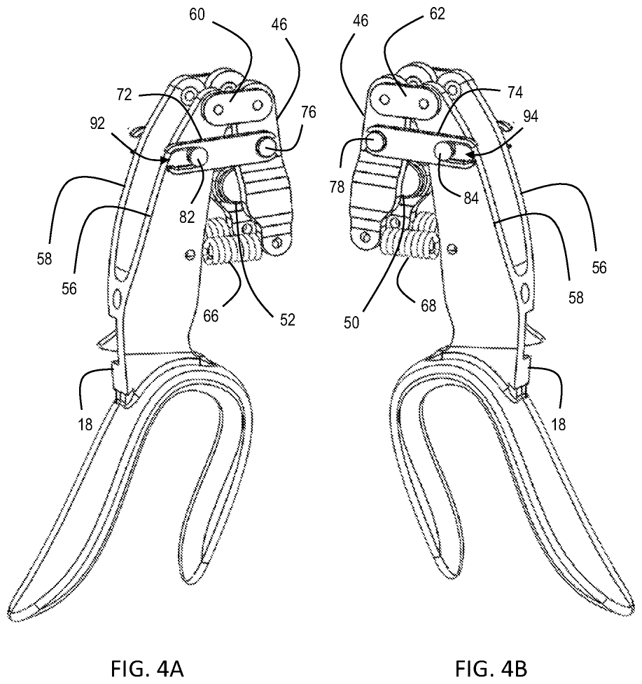

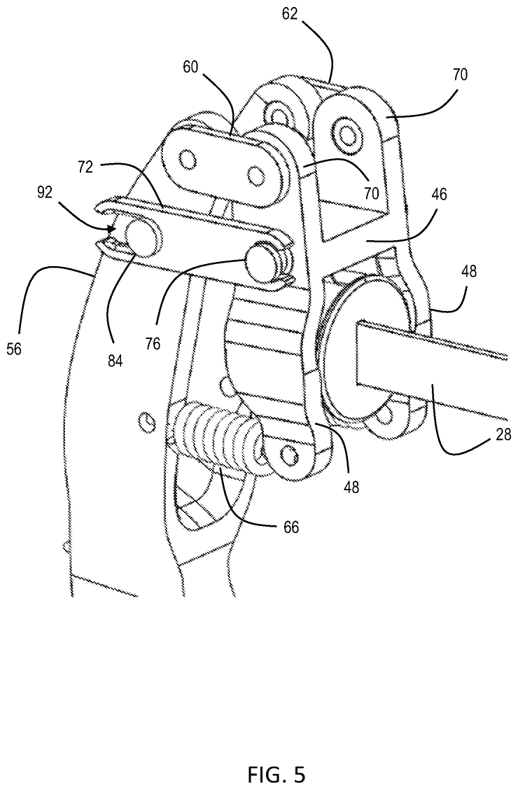

Lever 18 has an upper end positioned within body 26 and forming a pair of opposing tines 56 and 58 that extend on either side of longitudinal axis X-X. Each of opposing tines 56 and 58 is pivotally coupled to the upper ends of secondary lever 46 by one of a corresponding pair of links 60 and 62. A pair of springs 66 and 68 are coupled between the lower ends of secondary lever 46 and the base of tines 56 and 58, respectively. Springs 66 and 68 are configured to have a preload providing a predetermined biasing force that holds lever 18 and secondary lever 46 together so that pivoting of lever 18 by a user will pull secondary lever against proximal stop 44. A pair of positioning tabs 72 and 74 are pivotally connected to a second pair of posts 76 and 78 positioned on secondary lever 46 and to a first pair of posts 82 and 84 positioned on tines 56 and 58. As seen in FIGS. 4A, 4B, and 5, each of tabs 72 and 74 may be coupled to second set of posts 84 and 86 using slots 92 and 94 so that lever 18 can pivot relative to secondary lever 46 if the preload of springs 66 and 68 is overcome. Positioning tabs 72 and 74 maintain a minimum distance between lever 18 and secondary lever 46 against the preload force of springs 66 and 68 when a user is not applying a force to lever 18.

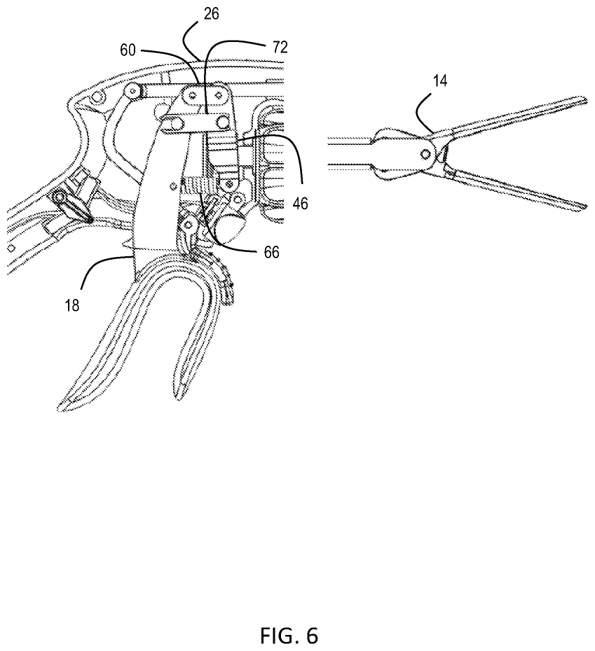

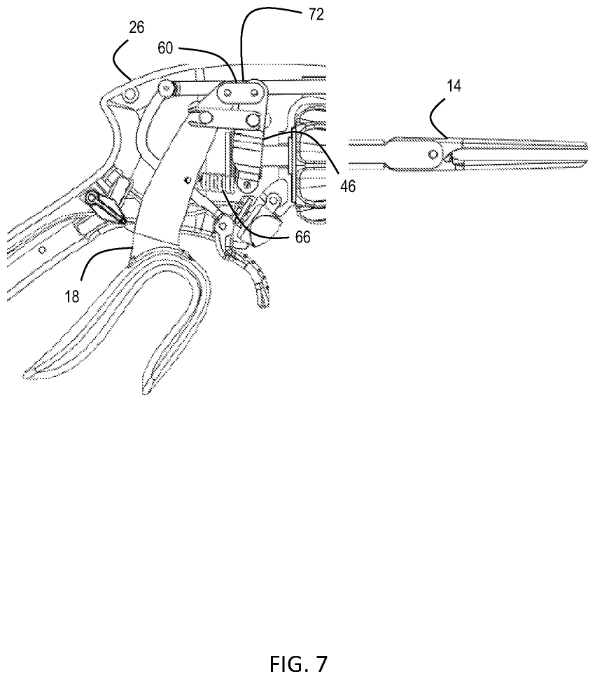

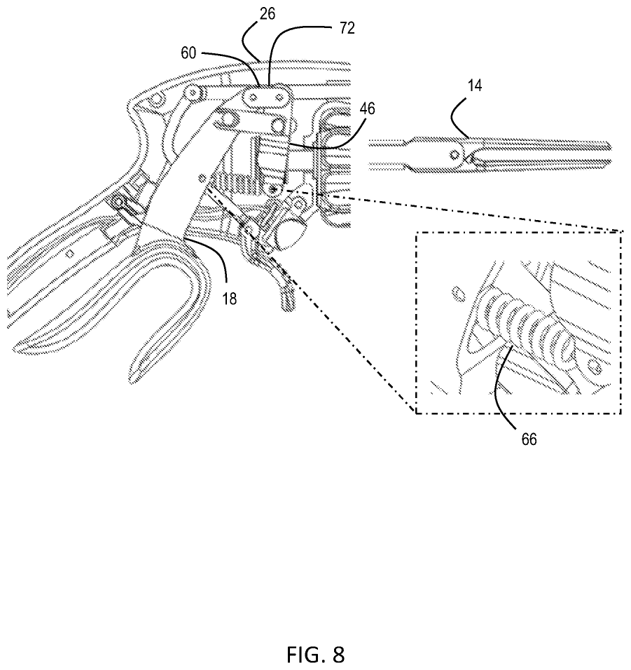

Referring to FIG. 6, when lever 18 is in a first position, jaws 14 will be open. As lever 18 is pivoted into a second position, as seen in FIG. 7, secondary lever 46 will pivot with lever 18 under the preload of springs 66 and 68. Pivoting of secondary lever 46 causes bearing surfaces 50 and 52 to push against proximal stop 44 thereby moving lever bearing 40 axially along longitudinal axis X-X so that jaws 14 are moved into the closed position. Referring to FIG. 8, any further application of force to lever 18 will overcome the preload of springs 66 and 68 so that spring 66 and 68 lengthen, thereby allowing lever 18 to pivot independently of secondary lever 46, which remains stationary. As a result, the further application of force is not transmitted to drive shaft 28.

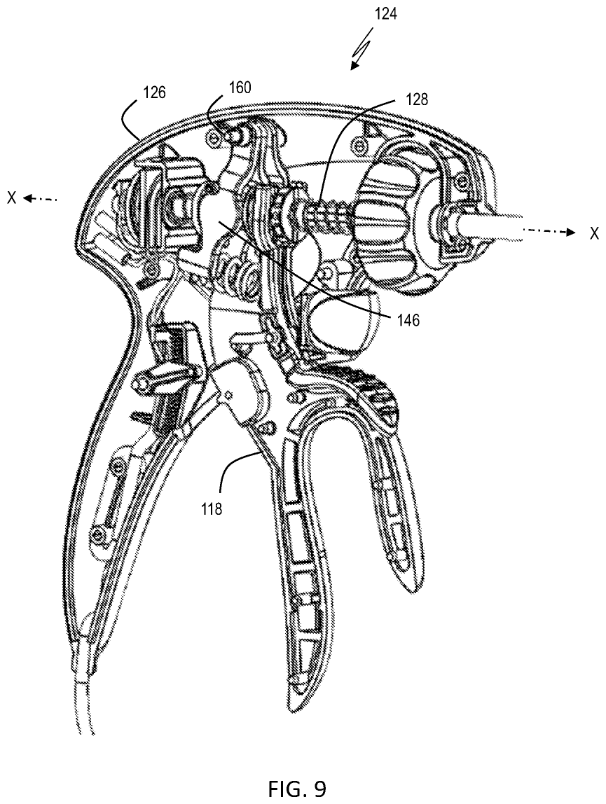

There is seen in FIG. 9, another embodiment of a handle assembly 124 comprised of a housing body 126 that encloses a drive shaft 128 that extends from body 126 and extends along longitudinal axis X-X of vessel sealer 12. In this embodiment, lever 118 is pivotally mounted at an upper end to a fixed location within housing body 126 and extends out of body 126 for grasping by a user. Lever 118 is coupled to drive shaft 128 via a secondary lever 146 that is mounted to a common pivot point and interconnected to lever 118 by a spring 166.

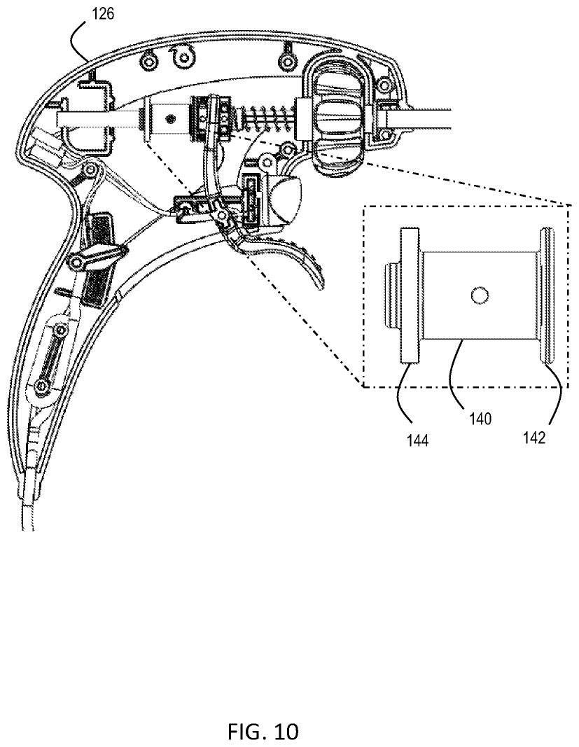

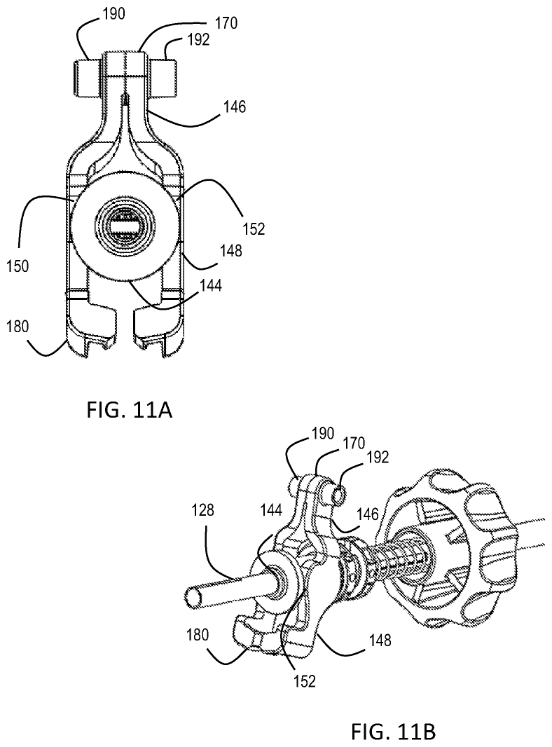

Referring to FIG. 10, a lever bearing 140 having a distal stop 142 and a proximal stop 144 is secured to drive shaft 128 so that movement of lever bearing 140 causes longitudinal translation of drive shaft 128 and thus opening and closing of jaws 14. As seen in FIGS. 11A and 11B, secondary lever 146 has an upper end 170 that can accept a pivot pin 160 for pivotally mounting secondary lever 146 within housing body 126. Upper end 170 may further including bearings 190 and 192 for pivotally coupling to lever 18 so that that lever 18 and secondary lever 146 are mounted to a common pivot point. Secondary lever 146 includes a pair of lower legs 148 that extend around lever bearing 140. Legs 148 includes proximal bearing surfaces 150 and 152 for engaging proximal stop 144. Pivotal movement of secondary lever 146 will cause proximal bearing surfaces 150 and 152 to move proximal stop 144 and lever bearing 140 axially, thereby moving drive shaft axially along axis X-X. Lower legs 148 cooperate to define a spring holder 180 for accepting spring 166.

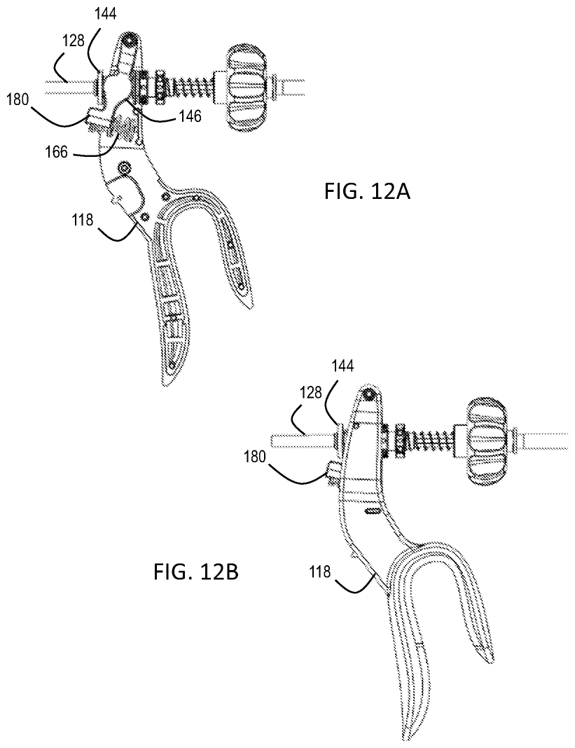

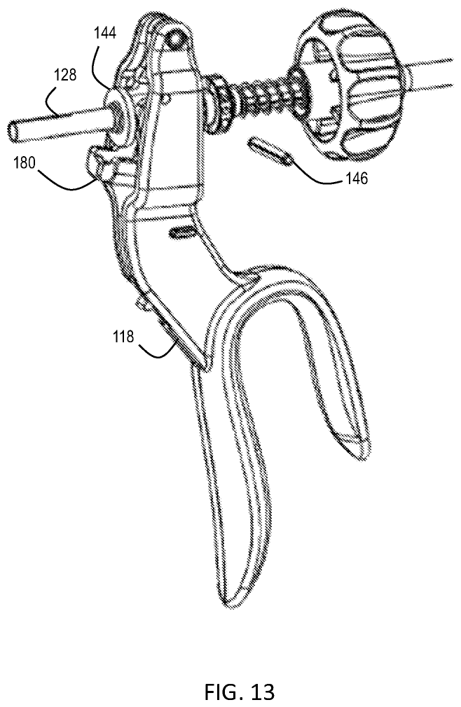

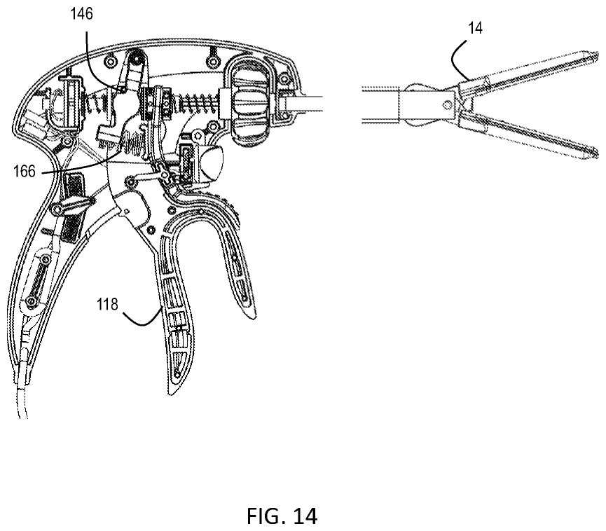

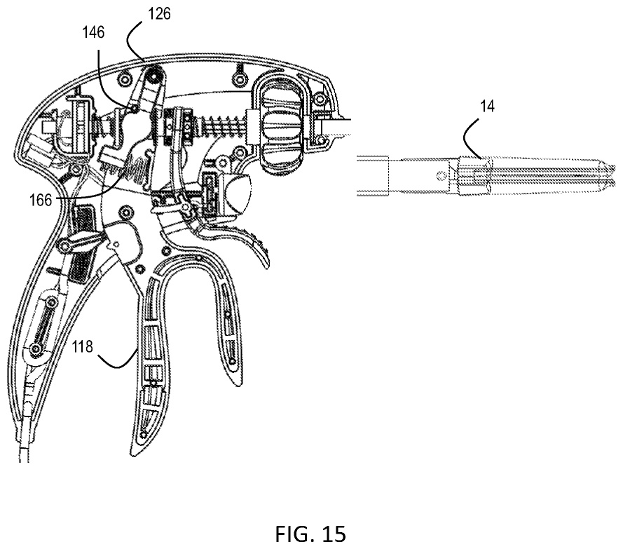

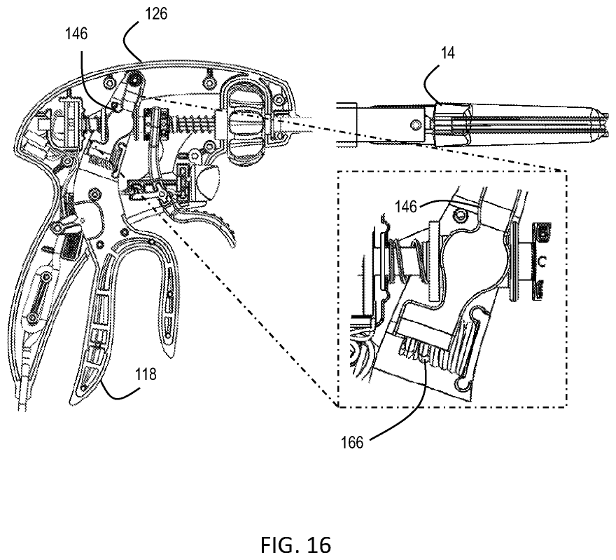

Referring to FIGS. 12A and 12B, lever 118 comprises two halves 118a and 118b that are closed about spring 166 and secondary lever 146 when handle body 126 is assembled. As seen in FIG. 13, a preload pin 182 may be used to partially compress spring 166 when installed so that spring will not compress when lever 118 is moved from the first position, where jaws 14 are open as seen in FIG. 14, to the second position, where jaws 14 are closed as seen in FIG. 15. Instead, spring 166 will only further compress when jaws 14 are already closed and a user continues to apply a force to lever 18, as seen in FIG. 16. As a result, any force applied to lever 118 after jaws have closed will be absorbed by spring 166 and not be communicated to jaws 14.

What is claimed is:

1. A surgical instrument, comprising:
a body having a drive shaft extending along a longitudinal axis and coupled to a pair of jaws that are moveable between an open and a closed position;
a lever bearing secured around the drive shaft for movement therewith and having a stop extending therefrom;
a first lever having a first upper end pivotally mounted within the body and a first lower end that extends out of the body;
a second lever having a second upper end interconnected to the first upper end of the first lever and extending to a second lower end, wherein the second lever engages the stop of the lever bearing; and
a spring extending between an intermediation portion of the first lever and the second lower end of the second lever.

2. The surgical instrument of claim 1, wherein the second lever includes pair of bearing surfaces in contact with the stop of the lever bearing

3. The surgical instrument of claim 2, further comprising a link pivotally interconnected to the first upper end of the first lever and to the second upper end of the second lever.

4. The surgical instrument of claim 3, wherein the second lever includes a pair of bearing surfaces in contact with the stop of the lever bearing.

5. The surgical instrument of claim 4, wherein the spring is configured to provide a force biasing the first lever and the second lever together.

6. The surgical instrument of claim 5, wherein the first lever is moveable between a first position where the first lever causes the second lever to position the lever bearing so that the drive shaft puts the jaws are in the open position, a second position where first lever causes the second lever to position the lever bearing position so that the drive shaft puts the jaws in the closed position, and a third position where the first lever has separated from the second lever against the bias of the spring so that the second lever and lever bearing do not move the drive shaft and the jaws remain in the closed position.

7. The surgical instrument of claim 6, further comprising a pair of tabs extending between a first set of posts positioned on the first lever and a second set of posts positioned in the second lever that set a predetermined minimum distance between the first lever and the second lever.

8. The surgical instrument of claim 2, wherein the first lever and the second lever are pivotally mounted within the body about a common pivot point.

9. The surgical instrument of claim 8, wherein the spring is configured to provide a force biasing the first lever and the second lever apart.

10. The surgical instrument of claim 9, wherein the first lever is moveable between a first position where the first lever causes the second lever to position that lever bearing so that the drive shaft puts the jaws are in the open position, a second position where first lever causes the second lever to position the lever bearing position so that the drive shaft puts the jaws in the closed position, and a third position where the first lever moved closer to the second lever against the bias of the spring so that the second lever and lever bearing do not move the drive shaft and the jaws remain in the closed position.

11. A method of limiting the amount of force applied by a user to the jaws of a surgical instrument, comprising the steps of:
providing a first lever extending from a body of the surgical instrument for movement by a user to close the jaws of the surgical instrument;
providing a second lever that is responsive to movement of the first lever to translate a lever bearing axially along a longitudinal axis of the body of the surgical instrument;
interconnecting the first lever and the second lever with a spring that allows for movement of the second lever along with the first lever when the first lever is moved to a position that results in closing of the jaws of the surgical instrument but prevents movement of the second lever when the first lever is moved beyond the closing of the jaws the second lever so that movement of the first lever past the position that results in closing of the jaws.

12. The method of claim 11, wherein the first lever and the second lever are interconnected a link that is pivotally connected to both the first lever and the second lever.

13. The method of claim 12, wherein the spring is configured to bias the first lever and the second lever toward each other.

14. The method of claim 13, wherein the first lever and the second lever are pivotally mounted in the body to a common pivot point.

15. The method of claim 14, wherein the spring is configured to bias the first lever and the second lever away from each other.
